(19) United States
(12) Patent Application Publication
(10) Pub. No.: US 2009/0221515 A1
(43) Pub. Date: Sep. 3, 2009

(54) MONOSACCHARIDE DERIVATIVES

(76) Inventors: Viswajanani Jitendra Sattigeri, Haryana (IN); Sudershan K. Arora, Maharashtra (IN); Venkata P. Palle, Haryana (IN); Mohammad Salman, Princeton, NJ (US); Abhijit Ray, Delhi (IN); Raj Kumar Shirumalla, Delhi (IN); Abdul Rehaman Abdul Rauf, Haryana (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 11/574,465

(22) PCT Filed: Aug. 31, 2005
(86) PCT No.: PCT/IB05/02573

§ 371 Date: Oct. 8, 2008

Related U.S. Application Data
(60) Provisional application No. 60/606,740, filed on Sep. 2, 2004.

(51) Int. Cl. A61K 31/7008; C07H 5/04

(52) U.S. Cl. 514/23; 536/18.7

(57) ABSTRACT

The present invention relates to monosaccharide derivatives as anti-inflammatory agents. The compounds disclosed herein can be useful for inhibition and prevention of inflammation and associated pathologies, including inflammatory and autoimmune diseases such as bronchial asthma, rheumatoid arthritis, type I diabetes, multiple sclerosis, allograft rejection, psoriasis, inflammatory bowel disease, ulcerative colitis, acne, atherosclerosis, cancer, pruritis or allergic rhinitis. Pharmacological compositions containing the compounds of the present invention and the methods of treating bronchial asthma, chronic obstructive pulmonary disease, rheumatoid arthritis, multiple sclerosis, type I diabetes, psoriasis, allograft rejection, inflammatory bowel disease, ulcerative colitis, acne, atherosclerosis, cancer, pruritis, allergic rhinitis and other inflammatory and/or autoimmune disorders, using the compounds are also provided.

### FIELD OF THE INVENTION

The present invention relates to monosaccharide derivatives as anti-inflammatory agents. The compounds disclosed herein can be useful for inhibition and prevention of inflammation and associated pathologies, including inflammatory and autoimmune diseases such as bronchial asthma, rheumatoid arthritis, type I diabetes, multiple sclerosis, allograft rejection psoriasis, inflammatory bowel disease, ulcerative colitis, acne, atherosclerosis, cancer, pruritis or allergic rhinitis. Pharmacological compositions containing the compounds disclosed herein and methods of treating bronchial asthma, chronic obstructive pulmonary disease, rheumatoid arthritis, multiple sclerosis, type I diabetes, psoriasis, allograft rejection; inflammatory bowel disease, ulcerative colitis, acne, atherosclerosis, cancer, pruritis, allergic rhinitis and other inflammatory and/or autoimmune disorders, using the compounds are also provided.

### BACKGROUND OF THE INVENTION

Inflammation is a key defense mechanism of the body that is activated as a result of tissue injury. The inflammatory process is self-containing, however, under certain pathophysiological conditions, the inflammatory process tends to perpetuate itself, giving rise to chronic inflammatory diseases like bronchial asthma, rheumatoid arthritis etc.

Although the exact cellular and molecular bases of most chronic inflammatory disease remain unclear, it has become apparent that several inflammatory cells act in concert towards initiation and perpetuation of an inflammatory response by releasing a wide range of chemokine, cytokine, proteolytic enzymes and other bioactive molecules. A case in point is mast cells primed by lymphocytes interact with environmental allergens and release mediators like histamine, prostaglandin, leukotrienes, etc. (Clin. Exp. Allergy, 32, 1682, 2002) to initiate an early inflammatory response. This is followed by a delayed inflammatory response due to release of cytokines (IL-4, IL-5, IL-6, IL-8, IL-13, GM-CSF and TNFalpha), chemokines and proteolytic enzymes (chymase, tryptase) (Chest 112, 523, 1997; Lancet 350, 59, 1997) that not only bring about tissue damage, but attract other inflammatory cells and initiate tissue fibrosis, and the cycle continues. Eosinophils infiltrate inflamed tissue following allergen-mast cell interaction in bronchial asthma and allergic rhinitis. Evidence is emerging that mast cells also interact with bacterial endotoxins leading to generation of cytokines like TNFalpha, that encourage neutrophil influx into the site of inflammation (Br. J. Pharmacol 123, 31 (1998); Br. J. Pharmacol 128, 700, (1999); Br. J. Pharmacol 136, 111, (2002); J. Clin. Invest., 109, 1351, 2002). Involvement of mast cells in the inflammatory response of chronic obstructive pulmonary disease (New Eng. J. Med., 347, 1040, 2002; Thorax 57, 649, 2002), inflammatory bowel disease (Gut, 45 Suppl II6, 1999) as well as rheumatoid arthritis (Science, 297, 1626, 2002), pathologies with prominent neutrophilic inflammation, has been proposed.

U.S. Pat. No. 6,329,344B1 discloses several monosaccharide derivatives described as cell adhesion inhibitors. It generally relates to substituted pentose and hexose monosaccharide derivatives, which are said to exhibit cell adhesion inhibitory and anti-inflammatory activities. U.S. Pat. No. 6,590,085B1 discloses several monosaccharide derivatives described as inhibitors of cell adhesion and cell adhesion mediated pathologies, including inflammatory and autoimmune diseases. U.S. Patent Application US 2002/0173632 A1 discloses furanose and amino furanose compounds said to be useful for rheumatoid, arthritis, immunomodulatory diseases inflammatory and proliferative diseases. U.S. Pat. No. 5,298,494 discloses derivatives of monosaccharides, which exhibit anti-proliferative and/or anti-inflammatory activity and are described as useful for treating mammals having inflammatory disorders and/or autoimmune disorders. U.S. Pat. No. 5,367,062 discloses derivatives of disubstituted and deoxydisubstituted α,D-lyxofuranosides which reportedly exhibit significant anti-inflammatory and antiproliferative activity and are said to be useful for treating inflammatory and/or autoimmune disorders. U.S. Pat. No. 5,360,794 discloses deoxydisubstituted or dideoxy disubstituted derivatives of α-D-mannofuranoside and β-L-gulofuranosides, which are said to exhibit anti-inflammatory and antiproliferative activity. U.S. Pat. No. 4,996,195 discloses derivatives of α,D-glucofuranose and α,D-allofuranose described as useful for treating animals and mammals with inflammatory and/or autoimmune disorders. U.S. Pat. No. 5,010,058 discloses derivatives of 1,2-O-iso-propylidene-α-D-glucofuranose described as useful for treating animals and mammals with inflammatory and/or autoimmune disorders.

WO 93/13117 and U.S. Pat. No. 5,360,792 discloses 5- or 6-deoxy hexose monosaccharides having a saturated nitrogen containing heterocycle described as useful as anti-proliferative and anti-inflammatory compounds. WO 94/28910 discloses 5,6-dideoxy-5-amino derivatives of idose and 6-deoxy-6-amino derivatives of glucose, which are said to exhibit immunomodulatory, anti-inflammatory and anti-proliferative activity. WO 94/11381 discloses derivatives of pentose monosaccharides described as anti-proliferative and anti inflammatory compounds.

### SUMMARY OF THE INVENTION

Monosaccharide derivatives, which can be used for the inhibition and prevention of cell adhesion and cell adhesion-mediated pathologies including inflammatory and autoimmune diseases such as bronchial asthma, rheumatoid arthritis, type I diabetes, multiple sclerosis, allograft rejection or psoriasis are provided.

Pharmaceutically acceptable salts, pharmaceutically acceptable solvates, enantiomers, diastereomers or N-oxides of these compounds having the same type of activity are also provided.

Pharmaceutical compositions containing the compounds, and which may also contain pharmaceutically acceptable carriers or diluents, which may be used for the treatment of inflammatory and autoimmune diseases such as bronchial asthma, rheumatoid arthritis, type I diabetes, multiple sclerosis, allograft rejection, psoriasis inflammatory bowel disease, ulcerative colitis, acne, atherosclerosis, cancer, pruritis and allergic rhinitis are also provided.

Other aspects will be set forth in accompanying description, which follows, and in part will be apparent from the description or may be learnt by the practice of the invention.

In accordance with one aspect, there are provided compounds having a structure of Formula I

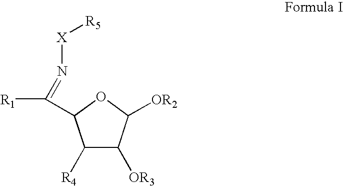

wherein

### X can be O, or NH.

R₁ can be hydrogen or methyl.
R₂ and R₃ can together form a five-membered acetal, wherein the carbon joining the oxygens is substituted with R₁ and Rₘ [wherein R₁ and Rₘ are independently selected from hydrogen, alkyl, alkenyl, alkynyl, cycloalkyl, aryl, or aralkyl; or R₁ and Rₘ can together join to form a 3- to 8-membered ring, wherein the ring may optionally contain one or more heteroatoms selected from O, N or S, and the ring may be optionally substituted with one or more of alkyl, alkenyl, alkynyl, acyl, substituted amino, cycloalkyl, or —C(═O)QR₇ (wherein Q can be O or NH, and R₇ can be selected from alkyl, alkenyl, alkynyl, aryl, aralkyl, cycloalkyl, heteroaryl, and when Q is NH only then can R₇ also be selected from heteroarylalkyl, heterocyclyl, heterocyclylalkyl, carboxy, oxo, hydroxyl, alkoxy, aryloxy, halogen (F, Cl, Br, I), aryl, aralkyl, heteroaryl, heterocyclyl, heteroarylalkyl, or heterocyclylalkyl), or R₁ and Rₘ can together join to form an oxo].
R₄ can be hydrogen, or OR_{c} (wherein R_{c} is selected from alkyl, alkenyl, alkynyl, cycloalkyl, aryl, aralkyl, heteroaryl, heterocyclyl, heteroarylalkyl, or heterocyclylalkyl). Also, when R₄ is OR_{c}, R₃ and R_{c} may together form an acetal (wherein the acetal is as defined earlier) and then R₂ can be alkyl, alkenyl, alkynyl, cycloalkyl, aryl, aralkyl, heteroaryl, heterocyclyl, heteroarylalkyl, or heterocyclylalkyl. Also, R₂ and R₃, instead of forming an acetal as defined earlier, may optionally and independently be selected from lower (C₁-C₄) allyl, (CH₂)ₖ-aryl (wherein k is an integer from 1-4), acyl, —C(═R_{y})NHRₓ (wherein R_{y} is O or S and Rₓ is alkyl, alkenyl, alkynyl, cycloalkyl, aryl, aralkyl, heteroaryl, heterocyclyl, heteroarylalkyl, or heterocyclylalkyl), and the R₄ is as defined earlier. Also, R₃ and R_{c} (when R₄ is OR_{c}) instead of forming an acetal as defined earlier, may optionally and independently be selected from lower (C₁-C₄) alkyl, (CH₂)ₖ-aryl (wherein k is an integer from 1-4), —C(═R_{y})NHRₓ (wherein R_{y} is O or S and Rₓ is the same as defined earlier) or acyl, and then R₂ is as defined earlier.
R₅ may be alkyl, alkenyl, alkynyl, aryl, aralkyl, heteroaryl, heterocylyl, heteroarylalkyl, heterocyclylalkyl, or —(CH₂)ₙ(C═O)OR_{z} (wherein n is 1-4, and R_{z} is hydrogen, alkyl, aralkyl, aryl, or heteroarylalkyl), —(CH₂)ₙ(C═O)NRₐR_{b} [wherein n is same as defined earlier, and Rₐ and R_{b} are independently selected from hydrogen or R_{d}, (wherein R_{d} is alkyl, alkenyl, alkynyl, cycloalkyl, aryl, aralkyl, heteroaryl, heterocyclyl, heteroarylalkyl, or heterocyclylalkyl)]. Also Rₐ and R_{b}, together with the nitrogen atom carrying them, can be the N-terminus of an aminoacid or di-tetrapeptide.

When X is O, then R₅ can be acyl or —C(═O)NR_{f}R_{q} [wherein R_{f} and R_{q} can be independently selected from hydrogen, alkyl, alkenyl, alkynyl, cycloalkyl, aryl, aralkyl, heterocyclyl, heteroaryl, heteroarylalkyl, heterocyclylalkyl, or S(O)₂R₆ (wherein R₆ can be selected from alkyl, alkenyl, alkynyl, cycloalkyl, aryl, aralkyl, heteroaryl, heterocyclyl, heterocyclylalkyl, heteroarylalkyl, or substituted amino)]. Also R_{f} and R_{q} can together form a ring.

When X is NH, then R₅ can be —C(═O)ORₛ (wherein Rₛ can be selected from alkyl, alkenyl, alkynyl, cycloalkyl, aryl, aralkyl, heterocyclylalkyl, or heteroarylalkyl), —YR_{d} (wherein Y is —C(═O), —C(═S) or SO₂ and R_{d} is same as defined earlier), or —C(=T)NRₜR_{f} (wherein Rₜ is OH or R_{f}, and T is O, S, —N(CN), —N(NO₂), or —CH(NO₂), and R_{f} is the same as defined earlier).

The following definitions apply to terms as used herein:

The term “alkyl” unless otherwise specified refers to a monoradical branched or unbranched saturated hydrocarbon chain having from 1 to 20 carbon atoms. This term can be exemplified by groups such as methyl, ethyl, n-propyl, iso-propyl, n-butyl, iso-butyl, t-butyl, n-hexyl, n-decyl, tetradecyl, and the like. Alkyl may further be substituted with one or more substituents selected from alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryloxy, aminosulfonyl, aminocarbonylamino, —COORₓ (wherein Rₓ is the same as defined earlier), —NHC(═O)R_{d}, —NR_{f}R_{q}, —C(═O)NR_{f}R_{q}, —NHC(═O)NR_{f}Rₜ, —N(OH)C(═O)NRₓRₜ, —C(═O)heteroaryl, C(═O)heterocyclyl, —O—C(═O)NR_{f}R_{q} wherein R_{d}, Rₜ, R_{f} and R_{q} are the same as defined earlier, nitro, —S(O)ₘR₆ wherein m is an integer from 0-2 and R₆ is the same as defined earlier. Unless otherwise constrained by the definition, all such alkyl substituents may be further substituted by 1-3 substituents chosen from alkyl, carboxy, —NR_{f}R_{q}, —C(═O)NR_{f}R_{q}, —O—C(═O)NR_{f}R_{q}, —NHC(═O)NR_{f}Rₜ (wherein R_{f}, R_{q} and Rₜ are the same as defined earlier), hydroxy, alkoxy, halogen, CF₃, cyano, and —S(O)ₘR₆, (wherein R₆ and m are the same as defined earlier); or an alkyl group as defined above may also be interrupted by 1-5 atoms of groups independently chosen from oxygen, sulfur and —NRₐ—, (where Rₐ is chosen from hydrogen, alkyl, cycloalkyl, alkenyl, cycloalkenyl, alkynyl, aryl, acyl, aralkyl, —C(═O)ORₛ wherein Rₛ is the same as defined earlier, S(O)₂R₆ where R₆ is as defined earlier, or —C(═O)NR_{f}R_{q} wherein R_{f} and R_{q} are as defined earlier). Unless otherwise constrained by the definition, all substituents may be further substituted by 1-3 substituents chosen from alkyl, carboxy, —NR_{f}R_{q}, —C(═O)NR_{f}R_{q}, —O—C(═O)NR_{f}R_{q} (wherein R_{f} and R_{q} are the same as defined earlier), hydroxy, alkoxy, halogen, CF₃, cyano, and —S(O)ₘR₆, (wherein m and R₆ are the same as defined earlier); or an alkyl group as defined above that has both substituents as defined above may also be interrupted by 1-5 atoms or groups as defined above.

The term “alkenyl” unless otherwise specified refers to a monoradical of a branched or unbranched unsaturated hydrocarbon group preferably having from 2 to 20 carbon atoms with cis or trans geometry. In the event that alkenyl is attached to the heteroatom, the double bond cannot be alpha to the heteroatom. Alkenyl may further be substituted with one or more substituents selected from alkyl, alkynyl, alkoxy, cycloalkyl, cycloalkenyl, acyl, acylamino, acyloxy, —COORₓ (wherein Rₓ is the same as defined earlier), —NHC(═O)R_{d}, —NR_{f}R_{q}, —C(═O)NR_{f}R_{q}, N(OH)C(═O)NRₓRₜ, —NHC(═O)NR_{f}Rₜ, —O—C(═O)NR_{f}R_{q} (wherein R_{f}, R_{q} and Rₜ are the same as defined earlier), alkoxycarbonylamino, azido, cyano, halogen, hydroxy, oxo, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, nitro, or S(O)ₘR₆ (wherein R₆ and m are the same as defined earlier). Unless otherwise constrained by the definition, all such alkyl substituents may optionally be further substituted by 1-3 substituents chosen from alkyl, carboxy, hydroxy, alkoxy, halogen, —CF₃, cyano, —NR_{f}R_{q}, —C(═O)NR_{f}R_{q}, —O—C(═O)NR_{f}R_{q} (wherein R_{f} and R_{q} are the same as defined earlier) and —S(O)ₘR₆, (wherein R₆ and m are the same as defined earlier).

The term “alkynyl” unless and otherwise specified refers to a monoradical of an unsaturated hydrocarbon, preferably having from 2 to 20 carbon atoms. In the event that alkynyl is attached to the heteroatom, the triple bond cannot be alpha to the heteroatom. Alkynyl may further be substituted with one or more substituents selected from alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, aralkyl, aryloxy, aminosulfonyl, aminocarbonylamino, nitro, heterocyclyl, heteroaryl, heterocyclyl alkyl, heteroarylalkyl, —COORₓ (wherein Rₓ is the same as defined earlier), —NHC(═O)R_{d}, —NR_{f}R_{q}, —NHC(═O)NR_{f}Rₜ, N(OH)C(═O)NRₓRₜ, —C(═O)NR_{f}R_{q}, —O—C(═O)NR_{f}R_{q} (wherein R_{d}, Rₜ, R_{f} and R_{q} are the same as defined earlier), or —S(O)ₘR₆ wherein R₆ and m are the same as defined earlier. Unless otherwise constrained by the definition, all such alkynyl substituents may optionally be further substituted by 1-3 substituents chosen from alkyl, carboxy, carboxyalkyl, hydroxy, alkoxy, halogen, CF₃, —NR_{f}R_{q}, —C(═O)NR_{f}R_{q}, —NHC(═O)NR_{f}Rₜ, —C(═O)NR_{f}R_{q} (wherein R_{d}, Rₜ, R_{f} and R_{q} are the same as defined earlier), cyano, and —S(O)ₘR₆, (wherein R₆ and m are the same as defined earlier).

The term “cycloalkyl” refers to cyclic alkyl groups of from 3 to 20 carbon atoms having a single cyclic ring or multiple condensed rings, which may optionally contain one or more olefinic bonds, unless or otherwise constrained by the definition. Such cycloalkyl groups include, by way of example, single ring structures such as cyclopropyl, cyclobutyl, cyclooctyl, cyclopentenyl, and the like, or multiple ring structures such as adamantanyl, and bicyclo [2.2.1]heptane, or cyclic alkyl groups to which is fused an aryl group, for example indane, and the like. Cycloalkyl may further be substituted with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —COORₓ (wherein Rₓ is the same as defined earlier), —NR_{f}R_{q}, —NHC(═O)NR_{f}Rₜ, —NHC(═O)R_{d}, N(OH)C(═O)NRₓRₜ, —C(═O)NR_{f}R_{q}, —O—C(═O)NR_{f}R_{q} (wherein R_{d}, Rₜ, R_{f} and R_{q} are the same as defined earlier), nitro, heterocyclyl, heteroaryl, heterocyclylalkyl, heteroarylalkyl, or S(O)ₘ—R₆ (wherein R₆ and m are the same as defined earlier). Unless otherwise constrained by the definition, all such cycloalkyl substituents may optionally be further substituted by 1-3 substituents chosen from alkyl, carboxy, hydroxy, alkoxy, halogen, CF₃, —NR_{f}R_{q}, —C(═O)NR_{f}R_{q}, —NHC(═O)NR_{f}Rₜ, —O—C(═O)NR_{f}R_{q} (wherein R_{d}, Rₜ, R_{f} and R_{q} are the same as defined earlier), cyano, and —S(O)ₘR₆, (wherein R₆ and m are the same as defined earlier).

The term “alkoxy” denotes the group O-alkyl wherein alkyl is the same as defined above.

The term “aralkyl” refers to alkyl-aryl linked through alkyl (wherein alkyl is the same as defined above) portion and the said allyl portion contains carbon atoms from 1-6 and aryl is as defined below. The examples of aralkyl groups are benzyl and the like.

The term “aryl” herein refers to a carbacyclic aromatic group, for example, phenyl, biphenyl or naphthyl ring and the like optionally substituted with 1 to 3 substituents selected from —(CH₂)_{w}C(═O)R_{g} (wherein w is an integer from 1-4 and R_{g} is hydroxyl), OR_{z}, NR_{f}R_{q}, —NHOR_{z}, —NHOH, halogen (F, Cl, Br, I), hydroxy, alkyl, alkenyl, alkynyl, cycloalkyl, alkoxy, acyl, aryloxy, cyano, nitro, —COORₓ (wherein Rₓ is the same as defined earlier), —NHC(═O)R_{d}, —NR_{f}R_{q}, N(OH)C(═O)NRₓRₜ, —C(═O)NR_{f}R_{q}, —NHC(—O)NR_{f}Rₜ, —(SO₂)ₘR₆ (wherein R₆, R_{d}, R_{f}, R_{q}, R_{z} and Rₜ and m are the same as defined earlier), carboxy, heterocyclyl, heteroaryl, heterocyclylalkyl, or heteroarylalkyl. The aryl group may optionally be fused with cycloalkyl group, wherein the cycloalkyl group may optionally contain heteroatoms selected from O, N, or S.

The term “aryloxy” denotes the group O-aryl wherein aryl is the same as defined above. The term “carboxy” as defined herein refers to —C(═O)OH.

The term “heteroaryl” unless otherwise specified refers to an aromatic ring structure containing 5 or 6 atoms, or a bicyclic aromatic group having 8 to 10 atoms, with one or more heteroatom(s) independently selected from N, O and S, optionally substituted with 1 to 3 substituent(s) selected from halogen (F, Cl, Br, I), hydroxy, alkyl, alkenyl, alkynyl, cycloalkyl, acyl, carboxy, aryl, alkoxy, aralkyl, cyano, nitro, —NR_{f}R_{q}, —(CH₂)_{w}C(═O)R_{g} (wherein w is an integer from 1-4 and R_{g} can be hydroxy, —COORₓ (wherein Rₓ is the same as defined earlier), OR_{z}, NR_{f}R_{q}, —NHOR_{z} or —NHOH, N(OH)C(═O)NRₓRₜ, —C(═O)NR_{f}R_{q} and —NHC(═O)NR_{f}Rₜ), —SO₂R₆, or —O—C(═O)NR_{f}R_{q} (wherein R₆, R_{z}, Rₜ, R_{d}, R_{f} and R_{q} are the same as defined earlier). Unless otherwise constrained by the definition, the substituents are attached to the ring atom, be it carbon or heteroatom. Examples of heteroaryl groups include pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like.

The term “heterocyclyl” unless otherwise specified, refers to a non-aromatic monocyclic or bicyclic cycloalkyl group having 5 to 10 atoms in which 1 to 3 carbon atoms in a ring are replaced by heteroatoms selected from O, S or N, and are optionally benzofused or fused heteroaryl of 5-6 ring members and/or are optionally substituted with halogen (F, Cl, Br, I), hydroxy, alkyl, alkenyl, alkynyl, cycloalkyl, acyl, aryl, alkoxy, alkaryl, cyano, nitro, oxo, carboxy, —C(═O)NR_{f}R_{q}, SO₂R₆, —O—C(═O)NR_{f}R_{q}, —NHC(═O)NR_{f}Rₜ, or —NR_{f}R_{q} (wherein Rₜ, R_{f} and R_{q} are the same as defined earlier). Unless otherwise constrained by the definition, the substituents are attached to the ring atom, be it carbon or heteroatom. Also unless otherwise constrained by the definition the heterocyclyl ring may optionally contain one or more olefinic bond(s). Examples of heterocyclyl groups include tetrahydrofuranyl, dihydrofuranyl, dihydropyridinyl, dihydrobenzofuryl, azabicyclohexyl, dihydroindolyl, piperidinyl or piperazinyl.

“Heteroarylalkyl” refers to alkyl-heteroaryl groups linked through the alkyl portion, wherein the alkyl and heteroaryl are the same as defined earlier. “Heterocyclylalkyl” refers to alkyl-heterocyclyl groups linked through the alkyl portion, wherein the alkyl and heterocyclyl are the same as defined earlier.

“Acyl” refers to —C(═O)R″ wherein R″ is selected from alkyl, cycloalkyl, aryl, aralkyl, heteroaryl, heterocyclyl, heteroarylalkyl or heterocyclylalkyl.

“Substituted amino” unless otherwise specified refers to —N(Rₖ)₂ wherein each Rₖ is independently selected from hydrogen (provided that both Rₖ groups are not hydrogen, defined as “amino”), alkyl, alkenyl, alkynyl, aralkyl, cycloalkyl, aryl, heteroaryl, heterocyclyl, heterocyclylalkyl, heteroarylalkyl, acyl, S(O)ₘR₆ (wherein m and R₆ is the same as defined above), —C(═R_{y})NR_{f}R_{q} (wherein R_{y}, R_{f} and R_{q} are the same as defined earlier) or NHC(═R_{y})NRₜR_{f} (wherein R_{y}, Rₜ and R_{f} are the same as defined earlier). Unless otherwise constrained by the definition, all amino substituents may optionally be further substituted by 1-3 substituents chosen from alkyl, aralkyl, cycloalkyl, aryl, heteroaryl, heterocyclyl, carboxy, carboxyalkyl, hydroxy, alkoxy, halogen, CF₃, cyano, —C(═R_{y})NR_{f}R_{q}, —O(C═O)NR_{f}R_{q} (wherein R_{f}, R_{q} and R_{y} are the same as defined earlier) —OC(—R_{y})NR_{f}R_{q}, or —S(O)ₘR₆, (wherein R₆ is the same as defined above and m is 0, 1 or 2).

The term “leaving group” generally refers to groups that exhibit the properties of being labile under the defined synthetic conditions and also, of being easily separated from synthetic products under defined conditions. Examples of such leaving groups include but are not limited to, halogen (F, Cl, Br, I), triflates, tosylate, mesylates, alkoxy, thioalkoxy, hydroxy radicals and the like.

The term “activated derivative of a carboxylic acid”, for example, that of a suitable protected amino acid, aliphatic acid or an aromatic acid, refers to the corresponding acyl halide (e.g., acid fluoride, acid chloride and acid bromide), corresponding activated esters (e.g. nitro phenyl ester, the ester of 1-hydroxybenzotriazole or the ester of hydroxysuccinimide, HOSu) or a mixed anhydride for example anhydride with ethyl chloroformate and other conventional derivatives within the skill of the art.

The term “protecting groups” refers to moieties which have the property of preventing specific chemical reaction at a site on the molecule undergoing chemical modification intended to be left unaffected by the particular chemical modification. Also the term protecting group, unless or other specified may be used with groups such as hydroxy, amino, carboxy and examples of such groups are found in T. W. Greene and P. G. M. Wuts, “Protective groups in Organic Synthesis”, 2^{nd} Ed, John Wiley and Sons, New York, N.Y., which is incorporated herein by reference. The species of the carboxylic protecting groups, amino protecting groups or hydroxy protecting group employed is not critical, so long as the derivatised moieties are stable to conditions of subsequent reactions and can be removed at the appropriate point without disrupting the remainder of the molecule.

“Amino acid” refers to both natural and unnatural amino acids. The term natural amino acid, as used herein, represents the twenty-two naturally-occurring amino acids glycine, alanine, valine, leucine, isoleucine, serine, methionine, threonine, phenylalanine, tyrosine, tryptophan, cysteine, proline, proline, histidine, aspartic acid, asparagines, glutamic acid, glutamine, γ-carboxyglutamic acid, arginine, ornithine and lysine in their L form. The term “unnatural amino acid”, as used herein, is intended to represent the ‘D’ form of the twenty-two naturally-occurring amino acids described above. It is further understood that the term unnatural amino acid includes homologues of the natural amino acids, and synthetically modified forms of the natural amino acids commonly utilized by those in the peptide chemistry arts when preparing synthetic analogues of naturally occurring peptides, including D and L forms. The synthetically modified forms include amino acids having alkylene chains shortened or lengthened by up to two carbon atoms, amino acids comprising optionally substituted aryl groups, and amino acids comprising halogenated groups, for example halogenated alkyl and aryl groups. The term “unnatural amino acids” as used herein also represents beta amino acids.

The term “peptide” refers to a molecule comprising a series of amino acids linked through amide bonds. Dipeptides comprise 2 amino acids, tripeptides comprise peptides having 3 amino acids, and tetrapeptide refers to peptides having four amino acids, wherein the term amino acid is as defined earlier.

The compounds disclosed herein contain one or more asymmetric carbon atoms and thus occur as racemates and racemic mixtures, single enantiomers, diastereomeric mixtures and individual diastereomers. All such isomeric forms of these compounds are expressly included herein. Each stereogenic carbon may be of the R or S configuration. Although the specific compounds exemplified in this application may be depicted in a particular stereochemical configuration, compounds having either the opposite stereochemistry at any given chiral center or mixtures thereof are envisioned as part of the invention. Although amino acids and amino acid side chains may be depicted in a particular configuration, both natural and unnatural forms are envisioned as part of the disclosure.

### DETAILED DESCRIPTION OF THE INVENTION

The compounds disclosed herein may be prepared by techniques well known in the art and familiar to the average synthetic organic chemist. In addition, particular compounds may be prepared, for example, by generally following the reaction scheme as depicted below. The compounds disclosed herein include both the syn and anti isomers either as pure isomers or as a mixture(s) thereof.

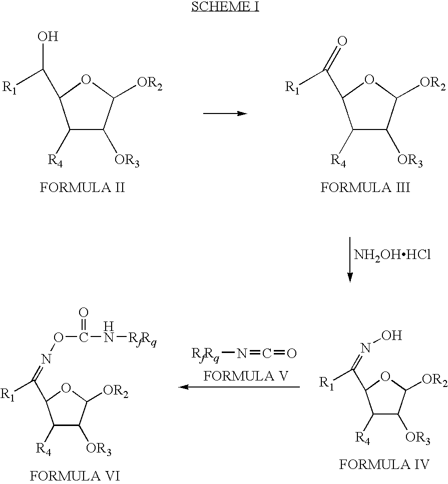

The compounds of Formula VI can be prepared following Scheme I. Thus a compound of Formula II (wherein R₁, R₂, R₃ and R₄ are the same as defined earlier) may be oxidized to furnish a compound of Formula III, which can react with hydroxylamine hydrochloride to furnish a compound of Formula IV, which can be reacted with compound of Formula V (wherein R_{f} and R_{q} are the same as defined earlier) to yield a compound of Formula VI.

The oxidation of the compound of Formula II to form a compound of Formula III can be carried out in an organic solvent, for example, dichloromethane, diethyl ether, or tetrahydrofuran, in the presence of oxidizing agents, such as pyridinium chlorochromate, pyridinium dichromate, dimethylsulphoxide with either oxalyl chloride or trifluoroacetic anhydride or acetic anhydride, or periodinane.

The condensation of the compound of Formula III with hydroxylamine hydrochloride to yield a compound of Formula IV can be carried out in an organic solvent, for example, ethanol, methanol, propanol or isopropyl alcohol, in the presence of a organic base, for example, pyridine, diisopropylethylamine, or triethylamine. When appropriately O-protected hydroxylamine is used in the above reaction one would obtain a compound of Formula IV where the OH is suitably substituted. Also, one skilled in the art in the field of this invention could use, for example, appropriately substituted hydrazine instead of hydroxylamine to obtain compounds described by Formula I where X is NH.

The reaction of compound of Formula IV with a compound of Formula V to yield a compound of Formula VI can be carried out in the presence of an organic solvent, for example, dichloromethane, dichloroethane, chloroform or carbontetrachloride.

Exemplary compounds prepared through Scheme I are listed here, and in Table I:
1-O-Dodecyl-2,3-O-isopropylidene-5,6-dideoxy-5-{[(2-fluoro-phenyl)-amino]-carbonyloxy}-imino-α-D-mannofuranoside (Compound No. 1),
1-O-Dodecyl-2,3-O-isopropylidene-5,6-dideoxy-5-{[(2,3-dichloro-phenyl)-amino]-carbonyloxy}-imino-α-D-mannofuranoside (Compound No. 2),
1-O-Dodecyl-2,3-O-isopropylidene-5,6-dideoxy-5-{[(2,6-difluoro-phenyl)-amino]-carbonyloxy}-imino-α-D-mannofuranoside (Compound No. 3),
1-O-Dodecyl-2,3-isopropylidene-5,6-dideoxy-5-{[(4-methoxy-phenyl)-amino]-carbonyloxy}-imino-α-D-mannofuranoside (Compound No. 4),
1-O-Dodecyl-2,3-O-isopropylidene-5,6-dideoxy-5-{[(4-chloro-phenyl)-amino]-carbonyloxy}-imino-α-D-mannofuranoside (Compound No. 5),
1-O-Dodecyl-2,3-O-isopropylidene-5,6-dideoxy-5-{[(4-methyl-phenyl)-amino]-carbonyloxy}-imino-α-D-mannofuranoside (Compound No. 6),
1-O-Dodecyl-2,3-O-isopropylidene-5,6-dideoxy-5-{[(4-fluoro-phenyl)-amino]-carbonyloxy}-imino-α-D-mannofuranoside (Compound No. 7),
1-O-Dodecyl-2,3-O-isopropylidene-5,6-dideoxy-5-{[(3-fluoro-phenyl)-amino]-carbonyloxy}-imino-α-D-mannofuranoside (Compound No. 8),
1-O-Dodecyl-2,3,4-isopropylidene-5,6-dideoxy-5-{[(4-chloro-2-methoxy-phenyl)-amino]-carbonyloxy}-imino-α-D-mannofuranoside (Compound No. 9),
1-O-Dodecyl-2,3-O-isopropylidene-5,6-dideoxy-5-{[(2-methyl-phenyl)-amino]-carbonyloxy}-imino-α-D-mannofuranoside (Compound No. 10),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 11),
1-O-Dodecyl-2,3-O-isopropylidene-5,6-dideoxy-5-{[(2-chloro-phenyl)-amino]-carbonyloxy}-imino-α-D-mannofuranoside (Compound No. 12),
1-O-Dodecyl-2,3-O-isopropylidene-5,6-dideoxy-5-{[(4-trifluoromethyl-phenyl)-amino]-carbonyloxy}-imino-α-D-mannofuranoside (Compound No. 13),
1-O-Dodecyl-2,3-O-isopropylidene-5,6-dideoxy-5-{[(2,4-dichloro-phenyl)-amino]-carbonyloxy}-imino-α-D-mannofuranoside (Compound No. 14),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 15),
1-O-Heptyl-2,3-O-isopropylidene-5-deoxy-5-{[(4-chloro-phenyl)-amino]-carbonyloxy}-imino-α-D-lyxofuranoside (Compound No. 16),
1-O-Heptyl-2,3-O-isopropylidene-5-deoxy-5-{[(4-methoxy-phenyl)-amino]-carbonyloxy}-imino-α-D-lyxofuranoside. (Compound No. 17),
1-O-Heptyl-2,3-O-isopropylidene-5-deoxy-5-{[phenyl-amino]-carbonyloxy}-imino-α-D-lyxofuranoside. (Compound No. 18),
1-O-Heptyl-2,3-O-isopropylidene-5-deoxy-5-{[(4-nitro-phenyl)-amino]-carbonyloxy}-imino-α-D-lyxofuranoside (Compound No. 19),
1-O-Heptyl-2,3-O-isopropylidene-5-deoxy-5-{[(4-chloro-phenyl)-sulphonylamino]-carbonyloxy}-imino-α-D-lyxofuranoside. (Compound No. 20),
1-O-Heptyl-2,3-O-isopropylidene-5-deoxy-5-{[phenyl-sulphonylamino]-carbonyloxy}-imino-α-D-lyxofuranoside (Compound No. 21),
1-O-Heptyl-2,3-O-isopropylidene-5-deoxy-5-{[(4-methyl-phenyl)-sulphonylamino]-carbonyloxy}-imino-α-D-lyxofuranoside (Compound No. 22),
1,2-O-Isopropylidene-3-O-decyl-5-deoxy-5-{[(4-chloro-phenyl)-sulphonylamino]-carbonyloxy}-imino-α-D-xylofuranoside (Compound No. 23),
1,2-O-Isopropylidene-3-O-decyl-5-deoxy-5-{[phenyl-sulphonylamino]-carbonyloxy}-imino-α-D-xylofuranoside (Compound No. 24),
1,2-O-Isopropylidene-3-O-decyl-5-deoxy-5-{[(4-methyl-phenyl)-sulphonylamino]-carbonyloxy}-imino-α-D-xylofuranoside (Compound No. 25),
1,2-O-Isopropylidene-3-O-decyl-5-deoxy-5-{[(4-chloro-phenyl)-amino]-carbonyloxy}-imino-α-D-xylofuranoside (Compound No. 26),
1,2-O-Isopropylidene-3-O-decyl-5-deoxy-5-{[phenyl-amino]-carbonyloxy}-imino-α-D-xylofuranoside (Compound No. 27),
1,2-O-Isopropylidene-3-O-decyl-5-deoxy-5-{[(4-nitro-phenyl-amino]-carbonyloxy}-imino-α-D-xylofuranoside (Compound No. 28),
1,2-O-Isopropylidene-3-O-decyl-5-deoxy-5-{[(4-methoxy-phenyl)-amino]-carbonyloxy}-imino-α-D-xylofuranoside (Compound No. 29).

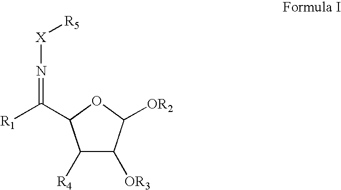

TABLE I
wherein when R₄ is OR_{c} then R₃ and R_{c} together form an
isopropylidene radical

| Com- | | | |
|---|---|---|---|
| pound | | | |
| No. | R₁ | R₂ | XR₅ |
| 1 | CH₃ | C₁₂H₂₅ | 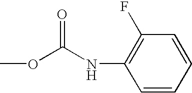 |
| 2 | CH₃ | C₁₂H₂₅ | 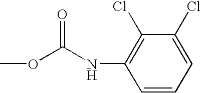 |
| 3 | CH₃ | C₁₂H₂₅ | 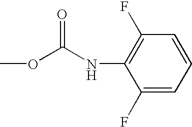 |
| 4 | CH₃ | C₁₂H₂₅ | 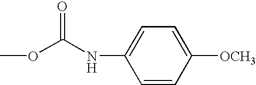 |
| 5 | CH₃ | C₁₂H₂₅ | 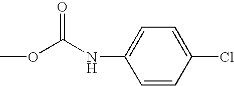 |
| 6 | CH₃ | C₁₂H₂₅ | 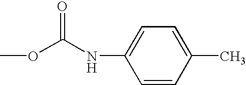 |
| 7 | CH₃ | C₁₂H₂₅ | 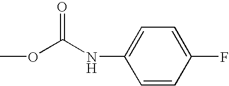 |
| 8 | CH₃ | C₁₂H₂₅ | 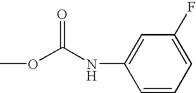 |
| 9 | CH₃ | C₁₂H₂₅ | 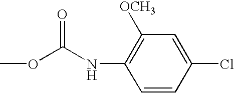 |
| 10 | CH₃ | C₁₂H₂₅ | 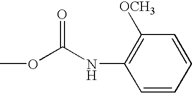 |
| 11 | CH₃ | C₁₂H₂₅ | 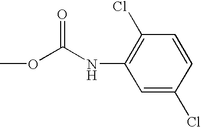 |
| 12 | CH₃ | C₁₂H₂₅ | 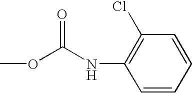 |
| 13 | CH₃ | C₁₂H₂₅ | 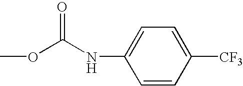 |
| 14 | CH₃ | C₁₂H₂₅ | 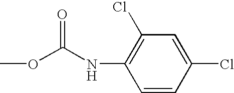 |
| 15* | CH₃ | C₁₂H₁₅ | 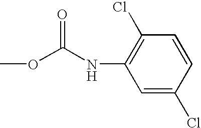 |
| 16 | H | C₇H₁₅ | 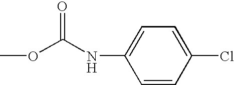 |
| 17 | H | C₇H₁₅ | 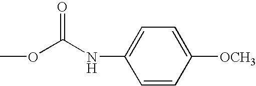 |
| 18 | H | C₇H₁₅ | 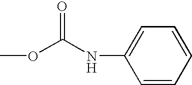 |
| 19 | H | C₇H₁₅ | 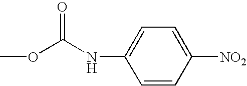 |
| 20 | H | C₇H₁₅ | 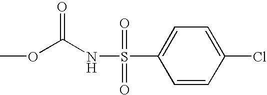 |
| 21 | H | C₇H₁₅ | 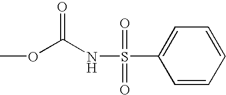 |
| 22 | H | C₇H₁₅ | 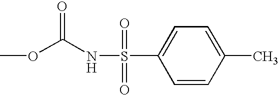 |

*refers to pure form of syn /anti isomer of compound No. 11

TABLE II
wherein when R₄ is OR_{c} and R₃ together form an isopropylidene radical.

| Com- | | | |
|---|---|---|---|
| pound | | | |
| No. | R₁ | R_{c} | XR₅ |
| 23 | H | C₁₀H₂₁ | 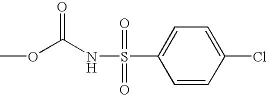 |
| 24 | H | C₁₀H₂₁ | 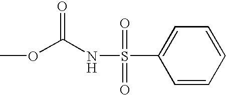 |
| 25 | H | C₁₀H₂₁ | 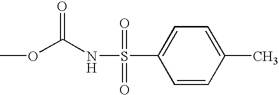 |
| 26 | H | C₁₀H₂₁ | 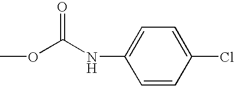 |
| 27 | H | C₁₀H₂₁ | 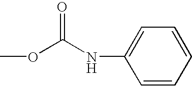 |
| 28 | H | C₁₀H₂₁ | 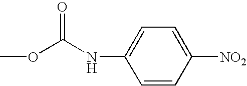 |
| 29 | H | C₁₀H₂₁ | 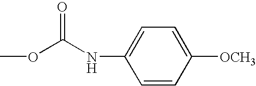 |

Examples set forth below demonstrate the general synthetic procedures for the preparation of representative compounds. The examples are provided to illustrate particular aspect of the disclosure and should not be constrained to limit the scope of the present invention.

### EXAMPLES

### Example 1

### Synthesis of 1-O-Dodecyl-2,3-O-isopropylidene-5,6-dideoxy-5-{[(2-fluoro-phenyl)-amino]-carbonyloxy}-imino-α-D-mannofuranoside (Compound No. 1)

### Step a: Synthesis of 1-O-Dodecyl-2,3-O-isopropylidene-5,6-dideoxy-5-oxo-α-D-mannofuranoside

To a solution of 1-O-dodecyl-2,3-O-isopropylidene-6-deoxy-α-D-mannofuranoside (synthesized following the procedure as described in U.S. Pat. No. 6,329,344) (1 g) in dichloromethane (12 ml) at room temperature was added pyridinium chlorochromate (2.3 g) and silica gel (2.3 g). The reaction mixture so obtained was refluxed for about 4 hours. The reaction mixture was then filtered over a pad of celite and the residue was washed with dichloromethane. The filtrate was washed with water, dried over anhydrous sodium sulphate and the residue purified over silica gel column using 13% ethyl acetate-hexane as eluent to furnish the title compound (1 gm).

### Step b: Synthesis of 1-O-Dodecyl-2,3-O-isopropylidene-5,6-dideoxy-5-hydroxy-imino-α-D-mannofuranoside

To a solution of the compound (1 g) obtained from step a above, in ethanol (8 ml) was added pyridine (8 ml) and hydroxylamine hydrochloride (560 mg). The reaction mixture was refluxed for about 5 hrs. The solvent was evaporated under reduced pressure and reaction mixture was poured in to water and extracted with ethyl acetate. The organic extract was washed with brine and dried over anhydrous sodium sulphate. The solvent was evaporated under the reduced pressure and the residue obtained was purified over silica gel column using 20% ethyl acetate-hexane as eluent to furnish the title compound (0.9 gm).

### Step c: Synthesis of 1-O-Dodecyl-2,3-O-isopropylidene-5,6-dideoxy-5-{[(2-fluoro-phenyl)-amino]-carbonyloxy}-imino-α-D-mannofuranoside (Compound No. 1)

To a solution of the compound (100 mg) obtained from step b in dichloromethane (5 ml) at 0° C. was added 2-fluoro-phenyl isocyanate (0.03 ml) and stirred the reaction mixture at room temperature for about 2 hours. Solvent was evaporated under reduced pressure and the residue obtained was purified over a silica gel column using 20% ethyl acetate-hexane as eluent to furnish the title compound (110 mg).

¹H NMR (CDCl₃, 300 MHz): δ 8.37 (1H, bs, NH), 8.18 (1H, t, J=9 Hz), 7.26-7.04 (3H, m), 5.30-4.59 (4H, m), 3.68-3.38 (2H, m), 2.16 (s) & 2.14 (s) [3H], 1.55 (2H, d, J=9 Hz), 1.46 (3H, d, J=6 Hz), 1.30-1.25 (21H, m), 0.87 (3H, t, J=6 Hz); LCMS: m/e=523 (M⁺+1).

Analogues of 1-O-Dodecyl-2,3-O-isopropylidene-5,6-dideoxy-5-{[(2-fluoro-phenyl)-amino]-carbonyloxy}-imino-α-D-mannofuranoside (Compound No. 1) described below were prepared by replacing with the appropriate sugar and the appropriate isocynate, as applicable in each case.
1-O-Dodecyl-2,3-O-isopropylidene-5,6-dideoxy-5-{[(2,3-dichloro-phenyl)-amino]-carbonyloxy}-imino-α-D-mannofuranoside (Compound No. 2),
1-O-Dodecyl-2,3-O-isopropylidene-5,6-dideoxy-5-{[(2,6-difluoro-phenyl)-amino]-carbonyloxy}-imino-α-D-mannofuranoside (Compound No. 3),
1-O-Dodecyl-2,3-O-isopropylidene-5,6-dideoxy-5-{[(4-methoxy-phenyl)-amino]-carbonyloxy}-imino-α-D-mannofuranoside (Compound No. 4),
1-O-Dodecyl-2,3-O-isopropylidene-5,6-dideoxy-5-{[(4-chloro-phenyl)-amino]-carbonyloxy}-imino-α-D-mannofuranoside (Compound No. 5),
1-O-Dodecyl-2,3-O-isopropylidene-5,6-dideoxy-5-{[(4-methylphenyl)-amino]-carbonyloxy}-imino-α-D-mannofuranoside (Compound No. 6),
1-O-Dodecyl-2,3-O-isopropylidene-5,6-dideoxy-5-{[(4-fluoro-phenyl)-amino]-carbonyloxy}-imino-α-D-mannofuranoside (Compound No. 7),
1-O-Dodecyl-2,3-O-isopropylidene-5,6-dideoxy-5-{[(3-fluoro-phenyl)-amino]-carbonyloxy}-imino-α-D-mannofuranoside (Compound No. 8),
1-O-Dodecyl-2,3-O-isopropylidene-5,6-dideoxy-5-{[(4-chloro-2-methoxy-phenyl)-amino]-carbonyloxy}-imino-α-D-mannofuranoside (Compound No. 9),
1-O-Dodecyl-2,3-O-isopropylidene-5,6-dideoxy-5-{[(2-methoxy-phenyl)-amino]-carbonyloxy}-imino-α-D-mannofuranoside (Compound No. 10),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 11),
1-O-Dodecyl-2,3-O-isopropylidene-5,6-dideoxy-5-{[(2-chloro-phenyl)-amino]-carbonyloxy}-imino-α-D-mannofuranoside (Compound No. 12),
1-O-Dodecyl-2,3-O-isopropylidene-5,6-dideoxy-5-{[(4-trifluoromethyl-phenyl)-amino]-carbonyloxy}-imino-α-D-mannofuranoside (Compound No. 13),
1-O-Dodecyl-2,3-O-isopropylidene-5,6-dideoxy-5-{[(2,4-dichloro-phenyl)-amino]-carbonyloxy}-imino-α-D-mannofuranoside (Compound No. 14),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 15),
1-O-Heptyl-2,3-O-isopropylidene-5-deoxy-5-{[(4-chloro-phenyl)-amino]-carbonyloxy}-imino-α-D-lyxofuranoside (Compound No. 16),
1-O-Heptyl-2,3-O-isopropylidene-5-deoxy-5-{[(4-methoxy-phenyl)-amino]-carbonyloxy}-imino-α-D-lyxofuranoside. (Compound No. 17),
1-O-Heptyl-2,3-O-isopropylidene-5-deoxy-5-{[phenyl-amino]-carbonyloxy}-imino-α-D-lyxofuranoside. (Compound No. 18),
1-O-Heptyl-2,3-O-isopropylidene-5-deoxy-5-{[(4-nitro-phenyl)-amino]-carbonyloxy}-imino-α-D-lyxofuranoside (Compound No. 19),
1-O-Heptyl-2,3-O-isopropylidene-5-deoxy-5-{[(4-chloro-phenyl)-sulphonylamino]-carbonyloxy}-imino-α-D-lyxofuranoside. (Compound No. 20),
1-O-Heptyl-2,3-O-isopropylidene-5-deoxy-5-{[phenyl-sulphonylamino]-carbonyloxy}-imino-α-D-lyxofuranoside (Compound No. 21),
1-O-Heptyl-2,3-O-isopropylidene-5-deoxy-5-{[(4-methyl-phenyl)-sulphonylamino]-carbonyloxy}-imino-α-D-lyxofuranoside (Compound No. 22),
1,2-O-Isopropylidene-3-O-decyl-5-deoxy-5-{[(4-chloro-phenyl)-sulphonylamino]-carbonyloxy}-imino-α-D-xylofuranoside (Compound No. 23),
1,2-O-Isopropylidene-3-O-decyl-5-deoxy-5-{[phenyl-sulphonylamino]-carbonyloxy}-imino-α-D-xylofuranoside (Compound No. 24),
1,2-O-Isopropylidene-3-O-decyl-5-deoxy-5-{[(4-methyl-phenyl)-sulphonylamino]-carbonyloxy}-imino-α-D-xylofuranoside (Compound No. 25),
1,2-O-Isopropylidene-3-O-decyl-5-deoxy-5-{[(4-chloro-phenyl)-amino]-carbonyloxy}-imino-α-D-xylofuranoside (Compound No. 26),
1,2-O-Isopropylidene-3-O-decyl-5-deoxy-5-{[phenyl-amino]-carbonyloxy}-imino-α-D-xylofuranoside (Compound No. 27),
1,2-O-Isopropylidene-3-O-decyl-5-deoxy-5-{[(4-nitro-phenyl)-amino]-carbonyloxy}-imino-α-D-xylofuranoside (Compound No. 28),
1,2-O-Isopropylidene-3-O-decyl-5-deoxy-5-{[(4-methoxy-phenyl)-amino]-carbonyloxy}-imino-α-D-xylofuranoside (Compound No. 29).

### Pharmacological Activity

The compounds disclosed herein are tested in one or both of the assays described herein. Standard assays are used to evaluate activity of compounds on inflammatory cells. Attenuation of agonist induced release of lipid mediators of neutrophil chemotaxis, leukotriene B4 (LTB4), is used to evaluate inhibitory effect on neutrophils.

### A23187 Induced LTB₄ Release

Venous blood was collected from healthy human donors using heparin as an anti-coagulant Neutrophils were isolated from freshly drawn blood after dextran sedimentation and ficoll separation (Eur J Biochem. 169, 175, 1987). 180 μl of the of neutrophil suspension (0.2×10⁶ cells/ml) was taken and added 19 μL of Hank's Buffer salt solution along with 1 μL of the test drug (200 times concentrated) in a 24-well plate and incubated at 37° C. for 1 hour. 3 minutes before the end of test compound incubation, 0.25 mM Ca⁺⁺/Mg⁺⁺ were added. Then, 0.3 μg/ml A23187 (Sigma Chem, USA) was added and incubated for further 10 min at 37° C. The reaction was stopped by adding 80 μL of cold methanol and centrifuged to remove cell debris (J Pharmacol Exp Ther. 297:267, 2001). The samples were analysed for LTB₄ release using LTB₄ ELISA kits (Assay Design Inc., USA). The amount of LTB₄ released was quantified and percent inhibition of LTB₄ release was calculated with respect to the difference between the A23187 stimulated and negative control cells, to compute IC₅₀ values. In vitro data obtained on particular compounds showed IC₅₀ values of at least about 30 μM.

### Assay for 5-Lipoxygenase Activity

In a 96 well UV-plate, 100 μl of phosphate buffer saline (PBS) containing DTT (200 μM), ATP (100 μM) and calcium chloride (100 μM) are added. To each well 0.5 μl of test drug (200 times concentrated) or vehicle is added, followed by 4 μl of recombinant 5-Lox (3 units/μl) and is incubated at 37° C. for 5 min. The reaction is initiated by adding 1 μl of 1 mM freshly prepared arachidonic acid and increase in absorbance is monitored at 236 nm for 10 min. (J Biol. Chem. 261:11512, 1986) A plot of absorbance verses time curve is prepared and area under curve (AUC) is computed for each well. Percent inhibition of AUC for different treatments is calculated with respect to the difference between the Arachidonic acid stimulated and negative control values, to compute IC₅₀ values.

We claim:

1. A compound having the structure of Formula I 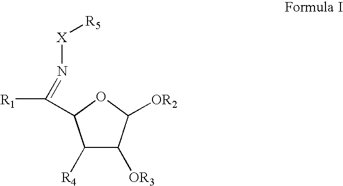
wherein
X is O, or NH;
R₁ is hydrogen or methyl;
R₂ and R₃ can together form a five-membered acetal, wherein the carbon joining the oxygens is substituted with R₁ and Rₘ [wherein R₁ and Rₘ are independently selected from hydrogen, alkyl, alkenyl, alkynyl, cycloalkyl, aryl, or aralkyl; or R₁ and Rₘ can together join to form a 3- to 8-membered ring, wherein the ring may optionally contain one or more heteroatoms selected from O, N or S, and the ring is optionally substituted with one or more of alkyl, alkenyl, alkynyl, acyl, substituted amino, cycloalkyl, or —C(═O)QR₇ (wherein Q is O or NH, and R₇ can be selected from alkyl, alkenyl, alkynyl, aryl, aralkyl, cycloalkyl, heteroaryl, and when Q is NH only then can R₇ also be selected from heteroarylalkyl, heterocyclyl, heterocyclylalkyl, carboxy, oxo, hydroxyl, alkoxy, aryloxy, halogen (F, Cl, Br, I), aryl, aralkyl, heteroaryl, heterocyclyl, heteroarylalkyl, or heterocyclylalkyl), or R₁ and Rₘ can together join to form an oxo];
R₄ is hydrogen, or OR_{c} (wherein R_{c} is selected from alkyl, alkenyl, alkynyl, cycloalkyl, aryl, aralkyl, heteroaryl, heterocyclyl, heteroarylalkyl, or heterocyclylalkyl), or, when R₄ is OR_{c}, R₃ and R_{c} may together form an acetal (wherein the acetal is as defined earlier) and then R₂ is alkyl, alkenyl, alkynyl, cycloalkyl, aryl, aralkyl, heteroaryl, heterocyclyl, heteroarylalkyl, or heterocyclylalkyl, or, R₂ and R₃, instead of forming an acetal as defined earlier, may optionally and independently be selected from lower (C₁-C₄) alkyl, (CH₂)ₖ-aryl (wherein k is an integer from 1-4), acyl, —C(—R_{y})NHRₓ (wherein R_{y} is O or S and Rₓ is alkyl, alkenyl, alkynyl, cycloalkyl, aryl, aralkyl, heteroaryl, heterocyclyl, heteroarylalkyl, or heterocyclylalkyl), and the R₄ is as defined earlier, or, R₃ and R_{c} (when R₄ is OR_{c}) instead of forming an acetal as defined earlier, may optionally and independently be selected from lower (C₁-C₄) alkyl, (CH₂)ₖ-aryl (wherein k is an integer from 1-4), —C(═R_{y})NHRₓ (wherein R_{y} is O or S and Rₓ is the same as defined earlier) or acyl, and then R₂ is as defined earlier,
R₅ is alkyl, alkenyl, alkynyl, aryl, aralkyl, heteroaryl, heterocylyl, heteroarylalkyl, heterocyclylalkyl, or —(CH₂)ₙ(C═O)OR_{z} (wherein n is 1-4, and R_{z} is hydrogen, alkyl, aralkyl, aryl, or heteroarylalkyl), —(CH₂)ₙ(C═O)NRₐR_{b} [wherein n is same as defined earlier, and Rₐ and R_{b} are independently selected from hydrogen or R_{d}, (wherein Rₐ is alkyl, alkenyl, alkynyl, cycloalkyl, aryl, aralkyl, heteroaryl, heterocyclyl, heteroarylalkyl, or heterocyclylalkyl)], or, Rₐ and R_{b}, together with the nitrogen atom carrying them, is the N-terminus of an aminoacid or di-tetrapeptide;
when X is O, then R₅ is acyl or —C(═O)NR_{f}R_{q} [wherein R_{f} and R_{q} can be independently selected from hydrogen, alkyl, alkenyl, alkynyl, cycloalkyl, aryl, aralkyl, heterocyclyl, heteroaryl, heteroarylalkyl, heterocyclylalkyl, or S(O)₂R₆ (wherein R₆ can be selected from alkyl, alkenyl, alkynyl, cycloalkyl, aryl, aralkyl, heteroaryl, heterocyclyl, heterocyclylalkyl, heteroarylalkyl, or substituted amino)], or; R_{f} and R_{q} can together form a ring; and
when X is NH, then R₅ is —C(═O)ORₛ (wherein Rₛ can be selected from alkyl, alkenyl, alkynyl, cycloalkyl, aryl, aralkyl, heterocyclylalkyl, or heteroarylalkyl), —YR_{d} (wherein Y is —C(═O), —C(═S) or SO₂ and R_{d} is same as defined earlier), or —C(=T)NRₜR_{f} (wherein Rₜ is OH or R_{f}, and T is O, S, —N(CN), —N(NO₂), or —CH(NO₂), and R_{f} is the same as defined earlier).

2. The compound of claim 1, wherein R₄ is OR_{c}, and R₃ and R_{c} together form isopropylidene.

3. The compound of claim 2, wherein R₂ is alkyl, alkenyl, alkynyl, cycloalkyl, aryl, aralkyl, heteroaryl, heterocyclyl, heterocyclylalkyl, or heteroarylalkyl, and XR₅ is —O(CO)NH-aryl, —O(CO)NH— substituted aryl, —O(CO)NH SO₂-aryl, or —O(CO)NH SO₂-substituted aryl.

4. The compound of claim 1, wherein R₄ is OR_{c} and R₂ and R₃ together form isopropylidene.

5. The compound of claim 4, wherein R_{c} is alkyl, alkenyl, alkynyl, cycloalkyl, aryl, aralkyl, heteroaryl, heterocyclyl, heterocyclylalkyl, or heteroarylalkyl, and XR₅ is —O(CO)NH-aryl, —O(CO)NH— substituted aryl, —O(CO)NH SO₂-aryl, or —O(CO)NH SO₂-substituted aryl.

6. A compound selected from:
1-O-Dodecyl-2,3-O-isopropylidene-5,6-dideoxy-5-{[(2-fluoro-phenyl)-amino]-carbonyloxy}-imino-α-D-mannofuranoside (Compound No. 1),
1-O-Dodecyl-2,3-O-isopropylidene-5,6-dideoxy-5-{[(2,3-dichloro-phenyl)-amino]-carbonyloxy}-imino-α-D-mannofuranoside (Compound No. 2),
1-O-Dodecyl-2,3-O-isopropylidene-5,6-dideoxy-5-{[(2,6-difluoro-phenyl)-amino]-carbonyloxy}-imino-α-D-mannofuranoside (Compound No. 3),
1-O-Dodecyl-2,3-O-isopropylidene-5,6-dideoxy-5-{[(4-methoxy-phenyl)-amino]-carbonyloxy}-imino-α-D-mannofuranoside (Compound No. 4),
1-O-Dodecyl-2,3-O-isopropylidene-5,6-dideoxy-5-{[(4-chloro-phenyl)-amino]-carbonyloxy}-imino-α-D-mannofuranoside (Compound No. 5),
1-O-Dodecyl-2,3-O-isopropylidene-5,6-dideoxy-5-{[(4-methyl-phenyl)-amino]-carbonyloxy}-imino-α-D-mannofuranoside (Compound No. 6),
1-O-Dodecyl-2,3-O-isopropylidene-5,6-dideoxy-5-{[(4-fluoro-phenyl)-amino]-carbonyloxy}-imino-α-D-mannofuranoside (Compound No. 7),
1-O-Dodecyl-2,3-O-isopropylidene-5,6-dideoxy-5-{[(3-fluoro-phenyl)-amino]-carbonyloxy}-imino-α-D-mannofuranoside (Compound No. 8),
1-Dodecyl-2,3-O-isopropylidene-5,6-dideoxy-5-{[(4-chloro-2-methoxy-phenyl)-amino]-carbonyloxy}-imino-α-D-mannofuranoside (Compound No. 9),
1-O-Dodecyl-2,3-O-isopropylidene-5,6-dideoxy-5-{[(2-methyl-phenyl)-amino]-carbonyloxy}-imino-α-D-mannofuranoside (Compound No. 10),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 11),
1-O-Dodecyl-2,3-O-isopropylidene-5,6-dideoxy-5-{[(2-chloro-phenyl)-amino]-carbonyloxy}-imino-α-D-mannofuranoside (Compound No. 12),
1-O-Dodecyl-2,3-O-isopropylidene-5,6-dideoxy-5-{[(4-trifluoromethyl-phenyl)-amino]-carbonyloxy}-imino-α-D-mannofuranoside (Compound No. 13),
1-O-Dodecyl-2,3-O-isopropylidene-5,6-dideoxy-5-{[(2,4-dichloro-phenyl)-amino]-carbonyloxy}-imino-α-D-mannofuranoside (Compound No. 14),
1-O-Dodecyl-2,3-O-isopropylidene-5,6-dideoxy-5-{[(2,5-dichloro-phenyl)-amino]-carbonyloxy}-imino-α-D-mannofuranoside (Compound No. 15),
1-O-Heptyl-2,3-O-isopropylidene-5-deoxy-5-{[(4-chloro-phenyl)-amino]-carbonyloxy}-imino-α-D-lyxofuranoside (Compound No. 16),
1-O-Heptyl-2,3-O-isopropylidene-5-deoxy-5-{[(4-methoxy-phenyl)-amino]-carbonyloxy}-imino-α-D-lyxofuranoside. (Compound No. 17),
1-O-Heptyl-2,3-O-isopropylidene-5-deoxy-5-{[phenyl-amino]-carbonyloxy}-imino-α-D-lyxofuranoside. (Compound No. 18),
1-O-Heptyl-2,3-O-isopropylidene-5-deoxy-5-{[(4-nitro-phenyl)-amino]-carbonyloxy}-imino-α-D-lyxofuranoside (Compound No. 19),
1-O-Heptyl-2,3-O-isopropylidene-5-deoxy-5-{[(4-chloro-phenyl)-sulphonylamino]-carbonyloxy}-imino-α-D-lyxofuranoside. (Compound No. 20),
1-O-Heptyl-2,3-O-isopropylidene-5-deoxy-5-{[phenyl-sulphonylamino]-carbonyloxy}-imino-α-D-lyxofuranoside (Compound No. 21),
1-O-Heptyl-2,3-O-isopropylidene-5-deoxy-5-{[(4-methyl-phenyl)-sulphonylamino]-carbonyloxy}-imino-α-D-lyxofuranoside (Compound No. 22),
1,2-O-Isopropylidene-3-O-decyl-5-deoxy-5-{[(4-chloro-phenyl)-sulphonylamino]-carbonyloxy}-imino-α-D-xylofuranoside (Compound No. 23),
1,2-O-Isopropylidene-3-O-decyl-5-deoxy-5-{[phenyl-sulphonylamino]-carbonyloxy}-imino-α-D-xylofuranoside (Compound No. 24),
1,2-O-Isopropylidene-3-O-decyl-5-deoxy-5-{[(4-methyl-phenyl)-sulphonylamino]-carbonyloxy}-imino-α-D-xylofuranoside (Compound No. 25),
1,2-O-Isopropylidene-3-O-decyl-5-deoxy-5-{[(4-chloro-phenyl)-amino]-carbonyloxy}-imino-α-D-xylofuranoside (Compound No. 26),
1,2-O-Isopropylidene-3-O-decyl-5-deoxy-5-{[phenyl-amino]-carbonyloxy}-imino-α-D-xylofuranoside (Compound No. 27),
1,2-O-Isopropylidene-3-O-decyl-5-deoxy-5-{[(4-nitro-phenyl]-amino]-carbonyloxy}-imino-α-D-xylofuranoside (Compound No. 28), or
1,2-O-Isopropylidene-3-O-decyl-5-deoxy-5-{[(4-methoxy-phenyl)-amino]-carbonyloxy}-imino-α-D-xylofuranoside (Compound No. 29).

7. A pharmaceutical composition comprising a compound of claim 1 and at least one pharmaceutically acceptable excipient.

8. A method of inhibiting or preventing inflammation, comprising administering a therapeutically effective amount of the composition of claim 7 to a patient in need thereof.

9. A method of inhibiting or preventing auto immune disease, comprising administering a therapeutically effective amount of the composition of claim 7 to a patient in need thereof.

10. A method of treating bronchial asthma, comprising administering a therapeutically effective amount of the composition of claim 7 to a patient in need thereof.

11. A method of treating chronic obstructive pulmonary disorder, comprising administering the pharmaceutical composition of claim 7 to a patient in need thereof.

12. A method of treating rheumatoid arthritis, comprising administering a therapeutically effective amount of the composition of claim 7 to a patient in need thereof.

13. A method of treating Type I diabetes, comprising administering a therapeutically effective amount of the composition of claim 7 to a patient in need thereof.

14. A method of treating multiple sclerosis, comprising administering a therapeutically effective amount of the composition of claim 7 to a patient in need thereof.

15. A method of treating allograft rejection, comprising administering a therapeutically effective amount of the composition of claim 7 to a patient in need thereof.

16. A method of treating psoriasis, comprising administering a therapeutically effective amount of the composition of claim 7 to a patient in need thereof.

17. A method of treating inflammatory bowel disease, comprising administering the pharmaceutical composition of claim 7 to a patient in need thereof.

18. A method of treating ulcerative colitis, comprising administering the pharmaceutical composition of claim 7 to a patient in need thereof.

19. A method of treating acne, comprising administering the pharmaceutical composition of claim 7 to a patient in need thereof.

20. A method of treating atherosclerosis, comprising administering the pharmaceutical composition of claim 7 to a patient in need thereof.

21. A method of treating cancer, comprising administering the pharmaceutical composition of claim 7 to a patient in need thereof.

22. A method of treating pruritis, comprising administering the pharmaceutical composition of claim 7 to a patient in need thereof.

23. A method of treating allergic rhinitis, comprising administering the pharmaceutical composition of claim 7 to a patient in need thereof.

24. A method of making a compound of Formula VI 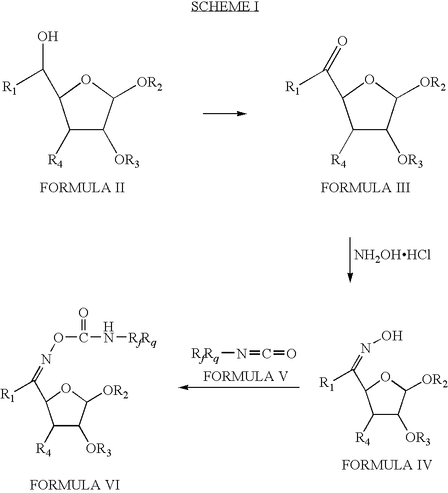
wherein
X is O, or NH;
R₁ is hydrogen or methyl;
R₂ and R₃ can together form a five-membered acetal, wherein the carbon joining the oxygens is substituted with R₁ and Rₘ [wherein R₁ and Rₘ are independently selected from hydrogen, alkyl, alkenyl, alkynyl, cycloalkyl, aryl, or aralkyl; or R₁ and Rₘ can together join to form a 3- to 8-membered ring, wherein the ring may optionally contain one or more heteroatoms selected from O, N or S, and the ring is optionally substituted with one or more of alkyl, alkenyl, alkynyl, acyl, substituted amino, cycloalkyl, or —C(═O)QR₇ (wherein Q is O or NH, and R₇ can be selected from alkyl, alkenyl, alkynyl, aryl, aralkyl, cycloalkyl, heteroaryl, and when Q is NH only then can R₇ also be selected from heteroarylalkyl, heterocyclyl, heterocyclylalkyl, carboxy, oxo, hydroxyl, alkoxy, aryloxy, halogen (F, Cl, Br, I), aryl, aralkyl, heteroaryl, heterocyclyl, heteroarylalkyl, or heterocyclylalkyl), or R₁ and Rₘ can together join to form an oxo];
R₄ is hydrogen, or OR_{c} (wherein R_{c} is selected from alkyl, alkenyl, alkynyl, cycloalkyl, aryl, aralkyl, heteroaryl, heterocyclyl, heteroarylalkyl, or heterocyclylalkyl), or, when R₄ is OR_{c}, R₃ and R_{c} may together form an acetal (wherein the acetal is as defined earlier) and then R₂ is alkyl, alkenyl, alkynyl, cycloalkyl, aryl, aralkyl, heteroaryl, heterocyclyl, heteroarylalkyl, or heterocyclylalkyl, or, R₂ and R₃, instead of forming an acetal as defined earlier, may optionally and independently be selected from lower (C₁-C₄) alkyl, (CH₂)ₖ-aryl (wherein k is an integer from 1-4), acyl, —C(═R_{y})NHRₓ (wherein R_{y} is O or S and Rₓ is alkyl, alkenyl, alkynyl, cycloalkyl, aryl, aralkyl, heteroaryl, heterocyclyl, heteroarylalkyl, or heterocyclylalkyl), and the R₄ is as defined earlier, or, R₃ and R_{c} (when R₄ is OR_{c}) instead of forming an acetal as defined earlier, may optionally and independently be selected from lower (C₁-C₄) alkyl, (CH₂)ₖ-aryl (wherein k is an integer from 1-4), —C(═R_{y})NHRₓ (wherein R_{y} is O or S and Rₓ is the same as defined earlier) or acyl, and then R₂ is as defined earlier;
R₅ is alkyl, alkenyl, alkynyl, aryl, aralkyl, heteroaryl, heterocylyl, heteroarylalkyl, heterocyclylalkyl, or —(CH₂)ₙ(C═O)OR_{z} (wherein n is 1-4, and R_{z} is hydrogen, alkyl, aralkyl, aryl, or heteroarylalkyl), —(CH₂)ₙ(C═O)NRₐR_{b} [wherein n is same as defined earlier, and Rₐ and R_{b} are independently selected from hydrogen or R_{d}, (wherein R_{d} is alkyl, alkenyl, alkynyl, cycloalkyl, aryl, aralkyl, heteroaryl, heterocyclyl, heteroarylalkyl, or heterocyclylalkyl)], or, Rₐ and R_{b}, together with the nitrogen atom carrying them, is the N-terminus of an aminoacid or di-tetrapeptide;
when X is O, then R₅ is acyl or —C(═O)NR_{f}R_{q} [wherein R_{f} and R_{q} can be independently selected from hydrogen, alkyl, alkenyl, alkynyl, cycloalkyl, aryl, aralkyl, heterocyclyl, heteroaryl, heteroarylalkyl, heterocyclylalkyl, or S(O)₂R₆ (wherein R₆ can be selected from alkyl, alkenyl, alkynyl, cycloalkyl, aryl, aralkyl, heteroaryl, heterocyclyl, heterocyclylalkyl, heteroarylalkyl, or substituted amino)], or, R_{f} and R_{q} can together form a ring; and
when X is NH, then R₅ is —C(═O)ORₛ (wherein Rₛ can be selected from alkyl, alkenyl, alkynyl, cycloalkyl, aryl, aralkyl, heterocyclylalkyl, or heteroarylalkyl), —YR_{d} (wherein Y is —C(═O), —C(═S) or SO₂ and R_{d} is same as defined earlier), or —C(=T)NRₜR_{f} (wherein Rₜ is OH or R_{f}, and T is O, S, —N(CN), —N(NO₂), or —CH(NO₂), and R_{f} is the same as defined earlier), comprising oxidizing a compound of Formula II to give a compound of Formula III;
reacting the compound of Formula III with hydroxylamine hydrochloride to give a compound of Formula IV; and
reacting the compound of Formula IV with a compound of Formula V (wherein R₇ and R₉ are as defined earlier) to give a compound of Formula VI.
